(19) United States
(12) Patent Application Publication
(10) Pub. No.: US 2015/0359554 A1
(43) Pub. Date: Dec. 17, 2015

(54) CELLULAR TISSUE DISSECTION METHOD

(71) Applicant: SEIKO EPSON CORPORATION, Tokyo (JP)

(72) Inventors: Hideki KOJIMA, Matsumoto-shi (JP); Toshiki ENDO, Sendai-shi (JP); Atsuhiro NAKAGAWA, Sendai-shi (JP); Tominaga TEIJI, Sendai-shi (JP)

(21) Appl. No.: 14/736,129

(22) Filed: Jun. 10, 2015

(30) Foreign Application Priority Data
Jun. 11, 2014 (JP) 2014-120269

(51) Int. Cl. A61B 17/3203

(52) U.S. Cl. CPC A61B 17/3203; A61B 2017/32032; A61B 2503/40

(57) ABSTRACT

A liquid is ejected to a cellular tissue, and the cellular tissue at a site where the liquid is ejected is dissected with an edge portion. The liquid is ejected in the form of a pulse flow. The liquid is ejected from a nozzle of a liquid ejection device. The edge portion is installed side by side with the nozzle. The site where the liquid is ejected is dissected with the edge portion.

This application claims the benefit of Japanese Patent Application No. 2014-120269, filed on Jun. 11, 2014. The content of the aforementioned application is incorporated herein by reference in its entirety.

### BACKGROUND

1. Technical Field

The present invention relates to a cellular tissue dissection method.

2. Related Art

Dissection processing is broadly employed in which a cellular tissue in a muscle, organ, tunic, blood vessel, tumor or the like is separated, severed and thus excised or stripped off. In this processing, a metallic surgical knife, electric surgical knife, laser surgical knife, water jet surgical knife or the like is used.

The water jet surgical knife dissects a cellular tissue with the impact energy of a water jet flow. The water jet surgical knife is able to dissect only the tissue without damaging any nerves, blood vessels or the like. JP-A-2008-82202 discloses a liquid ejection device as a water jet surgical knife. According to this, the liquid ejection device has a pulsation generator which applies pulsation to a fluid. The pulsating fluid is ejected to a cellular tissue, thus dissecting the cellular tissue.

If the connection between cells of a cellular tissue is strong, the cellular tissue is not separated unless a high-pressure pulse flow is ejected. If an extremely high-pressure pulse flow is ejected, there is a possibility that other cellular tissues than the target tissue may be damaged. Also, when pressure is adjusted, if the connection between cellular tissues is strong, dissection cannot be achieved unless the ejection is carried out for a long time. Thus, there is a demand for a cellular tissue dissection method that can easily dissect a cellular tissue.

### SUMMARY

An advantage of some aspects of the invention is to solve at least a part of the problems described above, and the invention can be implemented as the following forms or application examples.

### Application Example 1

This application example is directed to a cellular tissue dissection method including: ejecting a liquid to a cellular tissue; and dissecting, with a dissection tool, the cellular tissue at a site where the liquid is ejected.

According to this application example, the liquid is ejected to the cellular tissue. At the site where the liquid is ejected, the liquid is poured inside while a part of the cellular tissue is fractured. Thus, the distance between the cells is expanded, softening the tissue. Then, the cellular tissue at the site where the liquid is ejected is dissected with a dissection tool such as a cutting tool. At the softened site, since cutting with the dissection tool is made easier, the cellular tissue can be dissected easily.

### Application Example 2

In the cellular tissue dissection method according to the application example described above, the liquid may be ejected in the form of a pulse flow.

According to this application example, the liquid is ejected in the form of a pulse flow. With the pulse flow, the pressure fluctuation applied to the cellular tissue can be increased. As the pressure fluctuation increases, fatigue fracture can occur more easily in the part connecting cells together. Therefore, the cellular tissue can be softened easily.

### Application Example 3

In the cellular tissue dissection method according to the application example described above, the liquid may be ejected from a nozzle of a liquid ejection device. The dissection tool may be installed side by side with the nozzle. The site where the liquid is ejected may be dissected with the dissection tool.

According to this application example, the liquid ejection device has the nozzle for ejecting the liquid and the dissection tool such as a cutting tool. The surgical operator ejects the liquid to the cellular tissue from the nozzle of the liquid ejection device. Next, the surgical operator dissects, with the dissection tool, the cellular tissue at the site where the liquid is ejected. The dissection tool is installed side by side with the nozzle. Thus, the surgical operator need not hold the dissection tool and the liquid ejection device one after another and therefore can dissect the cellular tissue with high operability.

### BRIEF DESCRIPTION OF THE DRAWINGS

The invention will be described with reference to the accompanying drawings, wherein like numbers reference like elements.

FIG. 1A is a block diagram showing the configuration of a liquid ejection device according to a first embodiment. FIG. 1B is a partial schematic side view showing the structure of a nozzle in the liquid ejection device. FIG. 1C is a schematic view for explaining the behavior of a liquid in the nozzle.

FIG. 2A is a schematic cross-sectional view showing the internal configuration of a pulsation applying part. FIG. 2B is a graph showing transition of the volume of a liquid chamber.

FIG. 3 is a block diagram showing the electric control of the liquid ejection device.

FIGS. 4A to 4D are schematic views for explaining a dissection method for dissecting a cellular tissue.

FIGS. 5A to 5C are schematic views for explaining a dissection method for dissecting a cellular tissue.

FIGS. 6A to 6C are schematic views for explaining a dissection method for dissecting a cellular tissue.

FIG. 7 is a block diagram showing the configuration of a liquid ejection device according to a second embodiment.

FIG. 8 is a block diagram showing the configuration of a liquid ejection device according to a third embodiment.

FIGS. 9A to 9C are schematic views for explaining a dissection method for dissecting a cellular tissue.

### DESCRIPTION OF EXEMPLARY EMBODIMENTS

In the embodiments, a characteristic liquid ejection device and a characteristic example of a method for dissecting a cellular tissue using this liquid ejection device will be described with reference to the drawings. Hereinafter, the embodiments will be described with reference to the drawings. Each member in the drawings is illustrated not to scale, in order to show the member in a recognizable size in each drawing.

### First Embodiment

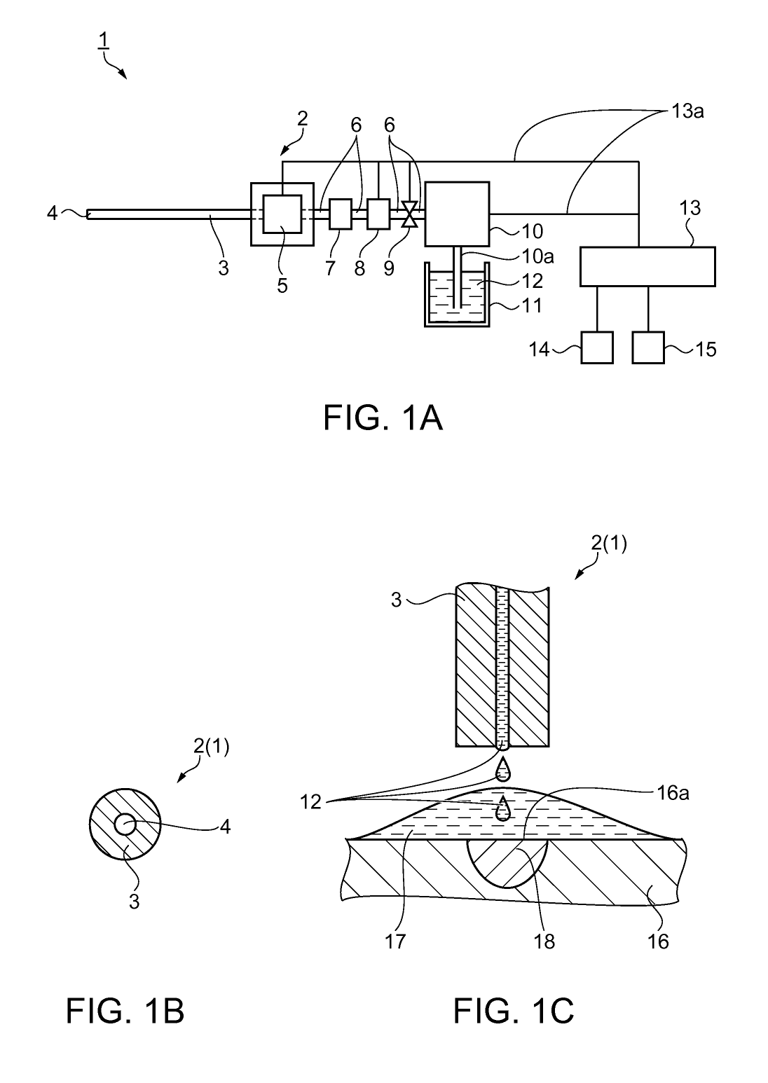

In this embodiment, a liquid ejection device as a surgical apparatus and a method for dissecting a cellular tissue using the liquid ejection device will be described with reference to FIGS. 1A to 6C. FIG. 1A is a block diagram showing the configuration of the liquid ejection device. FIG. 1B is partial schematic side view showing the structure of a nozzle in the liquid ejection device. The liquid ejection device 1 of this embodiment is a medical apparatus used in a medical institution and has the function of a surgical knife which ejects a fluid to an affected part and thus incises or excises the affected part. The liquid ejection device 1 can also be used for treatment and autopsy of other animals than humans.

As shown in FIG. 1A, the liquid ejection device 1 has a handpiece 2. The handpiece 2 is a device which the surgical operator holds in his/her hand and operates when carrying out a surgical operation. In other situations than surgical operation, the surgical operator is also referred to as an operator. An ejection tube 3 that is a channel for a fluid is installed in the handpiece 2. At one end of the ejection tube 3, a nozzle 4 as a liquid ejection opening for ejecting a fluid is installed. At the other end of the ejection tube 3, a pulsation applying part 5 is installed. A filter 7, a flowmeter 8, a solenoid valve 9, and a pump 10 are connected to the pulsation applying part 5 in this order via a tube 6. The pulsation applying part 5 is apart where the fluid passing through this part is turned into a pulse flow.

The filter 7 has the function of eliminating foreign matters, bacteria, air bubbles and the like contained in the fluid. The flowmeter 8 measures the flow rate of the fluid flowing through the tube 6. As the flowmeter 8, a hot-wire flowmeter, impeller flowmeter or the like can be used. The solenoid valve 9 is a valve controlled to open and close by an electrical signal. As the solenoid valve 9, a valve that opens and closes by a motor or electromagnet can be used.

As the pump 10, a syringe pump or tube pump can be used. In the case of a syringe pump, it is preferable that a device for supplying a fluid into the syringe is installed. This enables the liquid ejection device 1 to be driven continuously.

An inlet tube 10a is installed in the pump 10. One end of the inlet tube 10a is connected with a reservoir 11. There is a liquid 12 in the reservoir 11. As the liquid 12, for example, a physiological saline solution is used. The physiological saline solution is harmless to a living body and therefore can be used for a surgical operation.

The liquid ejection device 1 has a control device 13 as a control unit. The control device 13 controls the operations of the liquid ejection device 1. The pulsation applying part 5, the flowmeter 8, the solenoid valve 9 and the pump 10 are connected with the control device 13 via a cable 13a.

A main switch 14, an ejection switch 15 as a switch, and the like are installed on the control device 13. The main switch 14 is a switch for starting up the liquid ejection device 1. As the main switch 14 is turned on, electric power is supplied to the control device 13. The ejection switch 15 is a switch for switching between ejection and non-ejection of a fluid from the nozzle 4. The ejection switch 15 is a switch which the surgical operator steps on to operate.

As the surgical operator turns on the main switch 14, initial setting of the control device 13 is carried out. Then, the surgical operator turns on the ejection switch 15. The pump 10 is thus started up. The pump 10 causes the fluid 12 to flow to the solenoid valve 9. As the control device 13 opens the solenoid valve 9, the high-pressure liquid 12 in the form of a fluid advances into the tube 6. Then, the flowmeter 8 detects the flow rate of the fluid advancing through the tube 6 and outputs the detected flow rate to the control device 13.

The fluid advancing through the tube 6 passes through the filter 7. The filter 7 eliminates dust, air bubbles, salt crystals and the like from the liquid 12. To the liquid 12 reaching the pulsation applying part 5, pulse-like pulsation is applied by the pulsation applying part 5. A pulse-like pulsed flow is referred to as a pulse flow. The liquid 12 passing through the pulsation applying part 5 then passes through the ejection tube 3 and is ejected from the nozzle 4. The liquid 12 passing through the nozzle 4 is a pulse flow and therefore is ejected in the form of a pulse flow. As shown in FIG. 1B, the ejection tube 3 is a tube with the nozzle 4 located in the center. The liquid 12 in the form of a pulse flow is ejected from the nozzle 4.

FIG. 1C is a schematic view for explaining the behavior of the liquid in the nozzle. As shown in FIG. 1C, the surgical operator operates the handpiece 2 to move the nozzle 4 toward a cellular tissue 16. As the surgical operator turns on the ejection switch 15, the liquid 12 is ejected from the nozzle 4 and the liquid 12 collides with a collision point 16a on the cellular tissue 16. A liquid pool 17 of the accumulating liquid 12 is formed around the collision point 16a. Then, the cellular tissue 16 around the collision point 16a is softened by the impact. At the collision point 16a, a part of the cellular tissue 16 factures and the strength of connection between the cells is weakened. The tissue that is softened as a result of the pouring of the liquid inside and the expansion of the distance between the tissues, is referred to as a soft tissue 18. The soft tissue 18 can be easily cut with a dissection tool such as scissors.

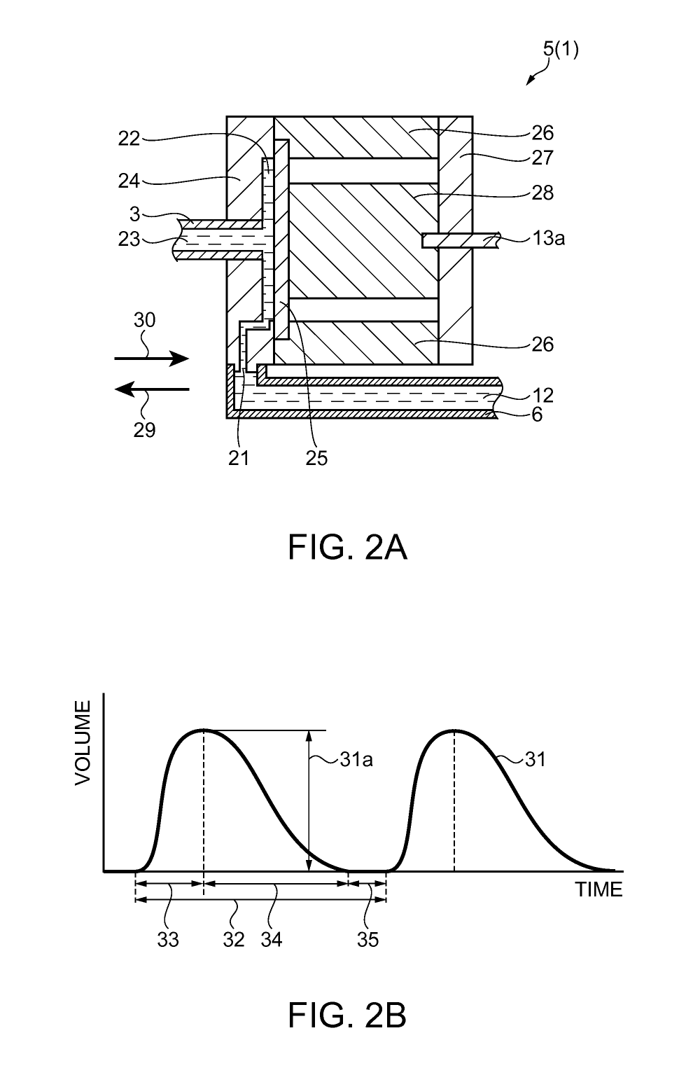

FIG. 2A is a schematic cross-sectional view showing the internal configuration of the pulsation applying part. In the pulsation applying part 5, an inlet channel 21, a liquid chamber 22 and an outlet channel 23 through which the liquid 12 supplied from the tube 6 passes are installed. The inlet channel 21 and the outlet channel 23 are formed in a first case 24. A diaphragm 25 is installed in such a way as to hold the liquid chamber 22 between the first case 24 and the diaphragm 25. The tube 6 is connected with the inlet channel 21. The ejection tube 3 is connected with the outlet channel 23.

On the right-hand side of the first case 24 in the illustration, a cylindrical second case 26 is installed in contact with the first case 24. The diaphragm 25 is a disk-shaped thin metal plate. An outer peripheral part of the diaphragm 25 is held and fixed between the first case 24 and the second case 26. On the right-hand side of the second case 26 in the illustration, a third case 27 is installed in contact with the second case 26. A piezoelectric element 28 as a volume varying unit, which is a multilayer piezoelectric element, is arranged between the diaphragm 25 and the third case 27. One end of the piezoelectric element 28 is fixed to the diaphragm 25. The other end thereof is fixed to the third case 27. The piezoelectric element 28 is connected with the control device 13 via the cable 13a.

As a drive voltage is applied from the control device 13, the piezoelectric element 28 changes the volume of the liquid chamber 22 formed between the diaphragm 25 and the first case 24. As the drive voltage applied to the piezoelectric element 28 rises, the piezoelectric element 28 expands, and the diaphragm 25 is pressed by the piezoelectric element 28 and thus flexes toward the liquid chamber 22, that is, in a first direction 29 in the illustration. As the diaphragm 25 flexes in the first direction 29, the volume of the liquid chamber 22 decreases. Then, the fluid inside the liquid chamber 22 is pushed out of the liquid chamber 22. The inner diameter of the outlet channel 23 is greater than the inner diameter of the inlet channel 21. That is, the fluid resistance in the outlet channel 23 is lower than the fluid resistance in the inlet channel 21. Since the inlet channel 21 is closer to the pump 10 than the outlet channel 23, the water pressure in the inlet channel 21 is higher than the water pressure in the outlet channel 23. Therefore, the major part of the fluid inside the liquid chamber 22 is pushed out of the liquid chamber 22 through the outlet channel 23.

Meanwhile, as the drive voltage applied to the piezoelectric element 28 falls, the piezoelectric element 28 contracts, and the diaphragm 25 is pulled by the piezoelectric element 28 and thus flexes toward the third case 27, that is, in a second direction 30 in the illustration. The piezoelectric element 28 contracts and the volume of the liquid chamber 22 increases. Therefore, the fluid is supplied into the liquid chamber 22 from the inlet channel 21.

The drive voltage applied to the piezoelectric element 28 repeats on-state (maximum voltage) and off-state (0 V) at a high frequency (for example, 300 Hz). Therefore, expansion and contraction of the volume of the liquid chamber 22 are repeated, thus applying pulsation to the fluid. The fluid pushed out of the liquid chamber 22 is ejected in the form of a pulse flow from the nozzle 4 at the end of the ejection tube 3. The ejection of a pulse flow means an ejection in the state where the flow rate or flow speed varies, and is not limited to repetition of ejection and stop of the fluid. That is, various other ejection forms are included, such as a form in which there is a complete interruption between ejections, or a form in which there is a low-pressure flow between ejections.

FIG. 2B is a graph showing the transition of the volume in the liquid chamber. In FIG. 2B, the vertical axis represents the volume of the liquid chamber 22. The volume decreases as it goes up in the graph. The horizontal axis represents the transition of time. Time shifts from the left to the right in the graph. A volume transition line 31 shows the volume of the liquid chamber 22 that is changed.

The volume transition line 31 is repeated on a cycle 32. One cycle 32 is divided into a rise section 33, a fall section 34, and a pause section 35. In the rise section 33, the volume transition line 31 has a shape similar to a sine waveform. At this time, a voltage is applied to the piezoelectric element 28 and the piezoelectric element 28 expands. Thus, the diaphragm 25 moves in the first direction 29 and the volume of the liquid chamber 22 decreases. Then, the liquid 12 in the liquid chamber 22 moves to the outlet channel 23.

In the fall section 34, the volume transition line 31 has a shape similar to a sine waveform. At this time, the voltage applied to the piezoelectric element 28 falls and the piezoelectric element 28 contracts. Thus, the diaphragm 25 moves in the second direction 30 and the volume of the liquid chamber 22 increases. Then, the liquid 12 flows into the liquid chamber 22 from the inlet channel 21. The fall section 34 is a longer time than the rise section 33. Thus, the liquid 12 flows out into the outlet channel 23 with great strength and flows in from the inlet channel 21 at a low speed. The pause section 35 is a section where the piezoelectric element 28 is maintained in the contracted state. Changing the length of the pause section 35 enables adjustment of the cycle 32.

The amount of change in the volume on the volume transition line 31 is defined as a volume change amount 31a. The volume change amount 31a can be adjusted as the control device 13 controls the piezoelectric element 28. In this manner, a pulse flow is formed in the pulsation applying part 5.

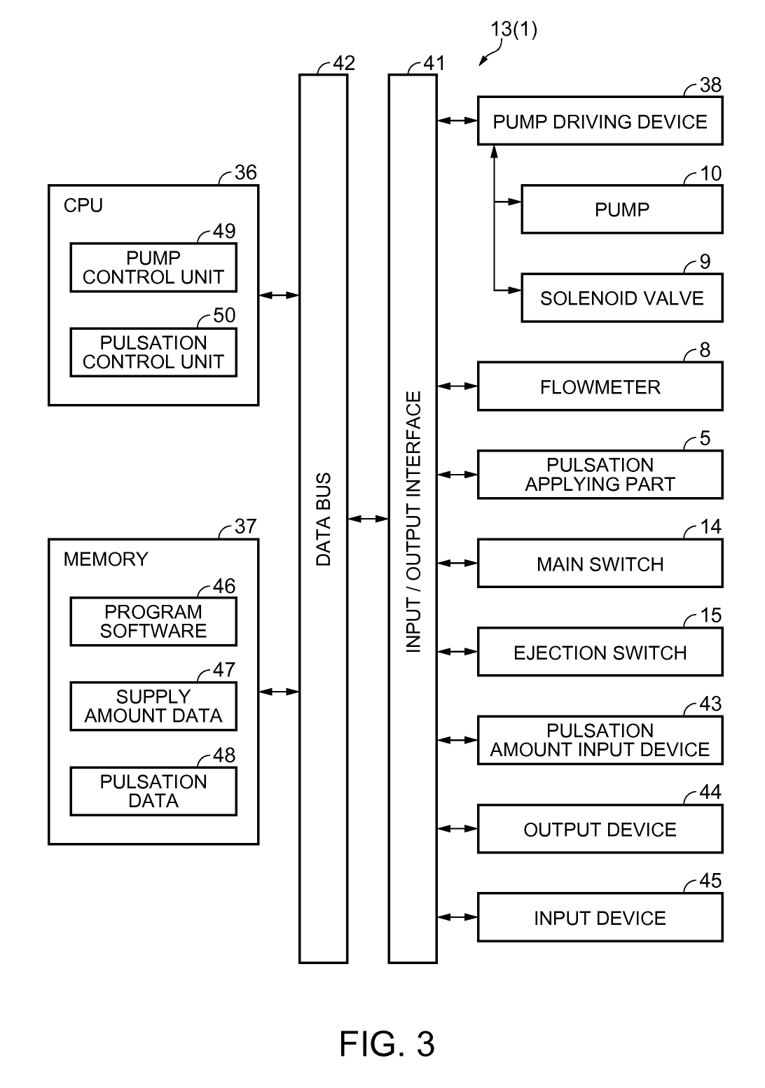

FIG. 3 is a block diagram showing the electric control of the liquid ejection device 1. In FIG. 3, the liquid ejection device 1 has the control device 13 for controlling the operations of the liquid ejection device 1. The control device 13 has a CPU 36 (central processing unit) which carries out various kinds of arithmetic processing as a processor, and a memory 37 which stores various kinds of information. A pump driving device 38, the flowmeter 8 and the pulsation applying part 5 are connected with the CPU 36 via an input/output interface 41 and a data bus 42. Moreover, the main switch 14, the ejection switch 15, a pulsation amount input device 43, an output device 44 and an input device 45 are connected with the CPU 36 via the input/output interface 41 and the data bus 42.

The pump driving device 38 is a device which drives the pump 10 and the solenoid valve 9. The pump driving device 38 takes in an instruction signal from the CPU 36. The pump driving device 38 then drives the pump 10 under the conditions of the pressure and flow rate indicated by the instruction signal. The pump driving device 38 also drives the solenoid valve 9 to open and close the valve.

The main switch 14 is a switch for starting up the liquid ejection device 1. As the main switch 14 is turned on, the control device 13 is started up. Then, as the surgical operator turns on the ejection switch 15, the pump 10 and the pulsation applying part 5 are driven. Then, after the water pressure of the liquid 12 rises, the solenoid valve 9 opens and the liquid 12 is ejected from the nozzle 4.

The pulsation amount input device 43 is a device with which the surgical operator inputs the amount of variance in the pulsation of the liquid 12. The pulsation amount input device 43 is, for example, a device for setting the volume change amount 31a of the liquid chamber 22. The pulsation amount input device 43 can be formed, for example, by a variable resistor, a circuit or switch for converting a resistance value of the variable resistor, and the like.

The output device 44 may include a liquid crystal display device, a light or speaker for giving a notification of abnormality, a device which carries out wired and wireless communication with an external computer, or the like. Thus, the control device 13 can display and output the state of the liquid ejection device 1 and the setting state set by the surgical operator.

The input device 45 may include a keyboard or mouse-type input device, a pen-type input device, and a device which carries out wired and wireless communication with an external computer. By such an input device 45, various data are inputted to the memory 37.

The memory 37 is a concept including a semiconductor memory such as RAM and ROM, and an external storage device such as hard disk and DVD-ROM. Functionally, a storage area for storing program software 46 describing procedures for controlling the operations of the liquid ejection device 1, and a storage area for storing supply amount data 47 that is data used when calculating the amount of the liquid 12 supplied, are set. In addition, a storage area for storing pulsation data 48 that is data about the pulsation of the liquid 12 is set. Moreover, a storage area that functions as a work area, temporary file or the like for the CPU 36, and various other storage areas are set.

The CPU 36 is configured to perform control to eject the liquid 12 from the nozzle 4 of the handpiece 2 according to the program software 46 stored in the memory 37. The CPU has a pump control unit 49 as a specific function implementation unit. The pump control unit 49 outputs an instruction signal to the pump driving device 38 and thus performs control to drive the pump 10 and cause the liquid 12 to flow. The pump control unit 49 takes in the flow rate of the liquid 12 detected by the flowmeter 8, and controls the flow rate of the liquid 12 to be ejected. The pump control unit 49 also opens and closes the solenoid valve 9 and thus controls the liquid 12 to flow and stop flowing.

The CPU 36 also has a pulsation control unit 50. The pulsation control unit 50 takes in the pulsation data 48 set by the pulsation amount input device 43, from the memory 37. The pulsation control unit 50 controls the piezoelectric element 28 of the pulsation applying part 5 and thereby controls the volume change amount 31a of the liquid chamber 22. As the liquid chamber 22 changes, the liquid 12 is ejected in the form of a pulse flow.

In this embodiment, each of the functions is realized by the program software, using the CPU 36. However, if each function can be realized by a standalone electronic circuit (hardware) without using the CPU 36, such an electronic circuit can be used.

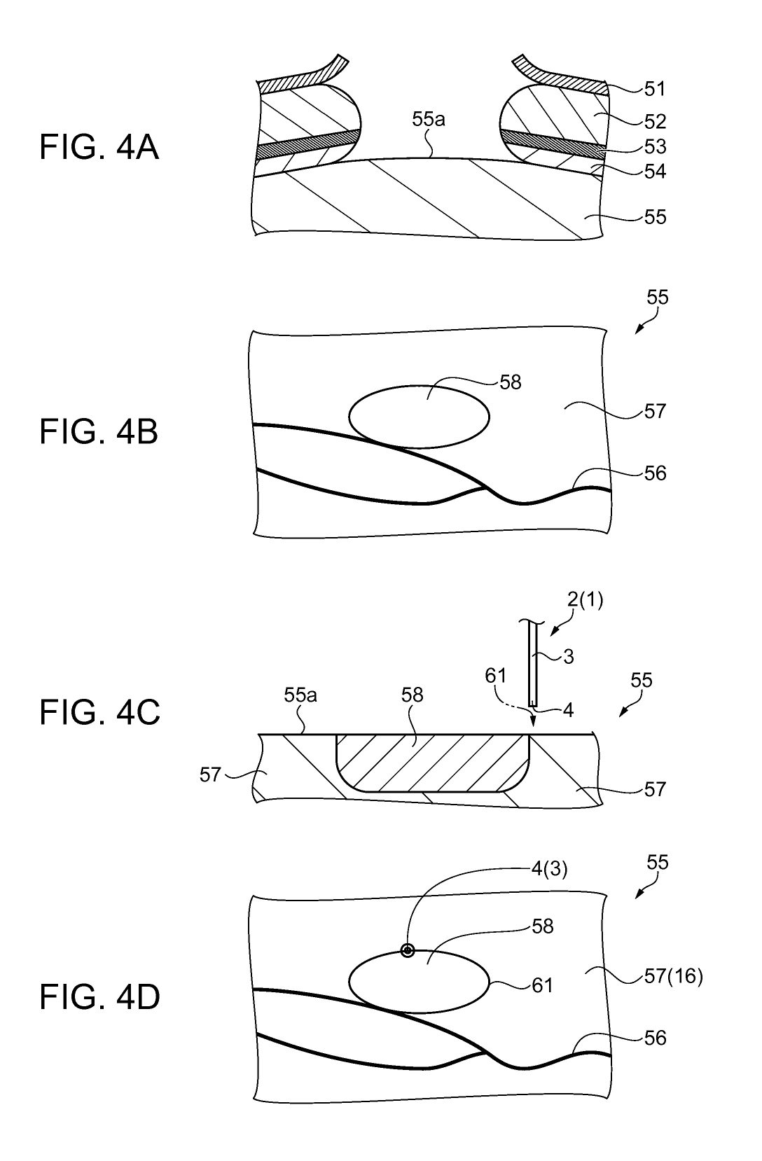

Next, a dissection method for dissecting the cellular tissue 16 using the liquid ejection device 1 will be described with reference to FIGS. 4A to 6C. FIGS. 4A to 6C are schematic views for explaining the dissection method for dissecting the cellular tissue. The site and type of the cellular tissue 16 are not particularly limited. However, here, for example, an example of dissecting a tumor from normal tissues on a brain surface is described. The brain surface refers to a surface of a cerebrum on the skull side. In a dissection procedure on the brain surface, it is necessary not to cause unwanted damage to normal tissues of important blood vessels, nerves and the like. There is a high risk of damaging normal tissues where the tissues are bonded together firmly at the boundary of the tissues.

FIGS. 4A and 4B show a step of exposing a dissection site. As shown in FIG. 4A, the surgical operator incises a head soft tissue 51. The head soft tissue 51 has a five-layer structure made up of an epidermis (scalp), a subcutaneous tissue, a galea, a subgaleal tissue, and a periosteum. Next, the surgical operator removes a part of a skull 52 and incises a meninx 53 and a pia mater 54. The meninx 53 is made up of a dura mater and an arachnoid.

Thus, brain surface 55a of a cerebrum 55 can be exposed. The cerebrum 55 is the cellular tissue 16 that is the target of dissection. As shown in FIG. 4B, blood vessels 56 extend through the cerebrum 55. In this example, a part of normal tissues 57 of the cerebrum 55 forms a tumor 58. One of the blood vessels 56 is very close to the tumor 58. In this step, the tumor 58 is exposed.

FIGS. 4C and 4D show the step of installing the nozzle 4 at the position of the cellular tissue 16 to be dissected. As shown in FIGS. 4C and 4D, a scheduled dissection line 61 is set, surrounding the tumor 58. That is, the scheduled dissection line 61 forms the boundary between the normal tissues 57 and the tumor 58. Then, the surgical operator installs the handpiece 2 in such a way that the nozzle 4 is situated at a position facing the scheduled dissection line 61. It is preferable that the handpiece 2 is installed in such a way that the direction of extension of the ejection tube 3 is in the direction of a normal line to the brain surface 55a. The ejected liquid 12 can be applied to the cerebrum 55 efficiently with strength.

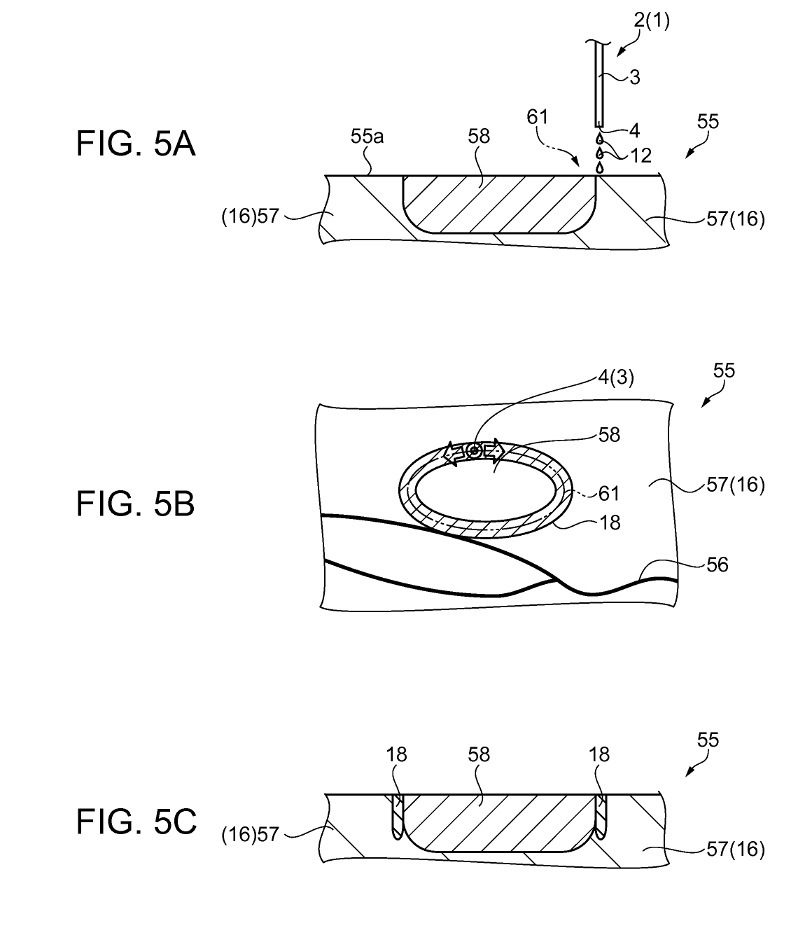

FIGS. 5A to 5C show the step of ejecting the liquid 12 to the cellular tissue 16 from the nozzle 4. As shown in FIGS. 5A and 5B, the surgical operator causes the liquid 12 to be ejected toward the cellular tissues 16 from the nozzle 4. Then, the surgical operator operates the handpiece 2 to move the nozzle 4 along the scheduled dissection line 61. The movement of the nozzle 4 may be one circular movement or a plurality of circular movements, and is not particularly limited. The direction of movement is not particularly limited, either, and may be one of clockwise movement and counterclockwise movement or a combination thereof.

The scheduled dissection line 61 is a line indicating the position where the tissues are to be split. The liquid 12 in the form of a pulse flow is ejected from the nozzle 4, and the liquid 12 is poured to the position where the tissues are to be split. As the liquid 12 is poured, a part of the cellular tissue 16 fractures and the distance between the tissues of the cellular tissue 16 expands. Thus, the cellular tissue 16 becomes the soft tissue 18, as shown in FIGS. 5B and 5C. The distance between the tumor 58 and the blood vessel 56 expands, which are very close to each other previously.

As the liquid 12 to be ejected, for example, a physiological saline solution is used. Thus, chemical damage to the cellular tissue 16 can be restrained. The ejection of the liquid 12 has a strength that is not high enough to dissect the cellular tissue 16. Even if the blood vessel 56 is close to the scheduled dissection line 61, the blood vessel 56 is not damaged because the strength of the ejection is not unnecessarily high.

The liquid 12 is ejected in the form of a pulse flow. Using the pulse flow enables increase in the pressure fluctuation applied to the cellular tissue 16. As the pressure fluctuation increases, fatigue fracture can occur more easily in the part that connects cells together. Therefore, the cellular tissue 16 becomes the soft tissue 18.

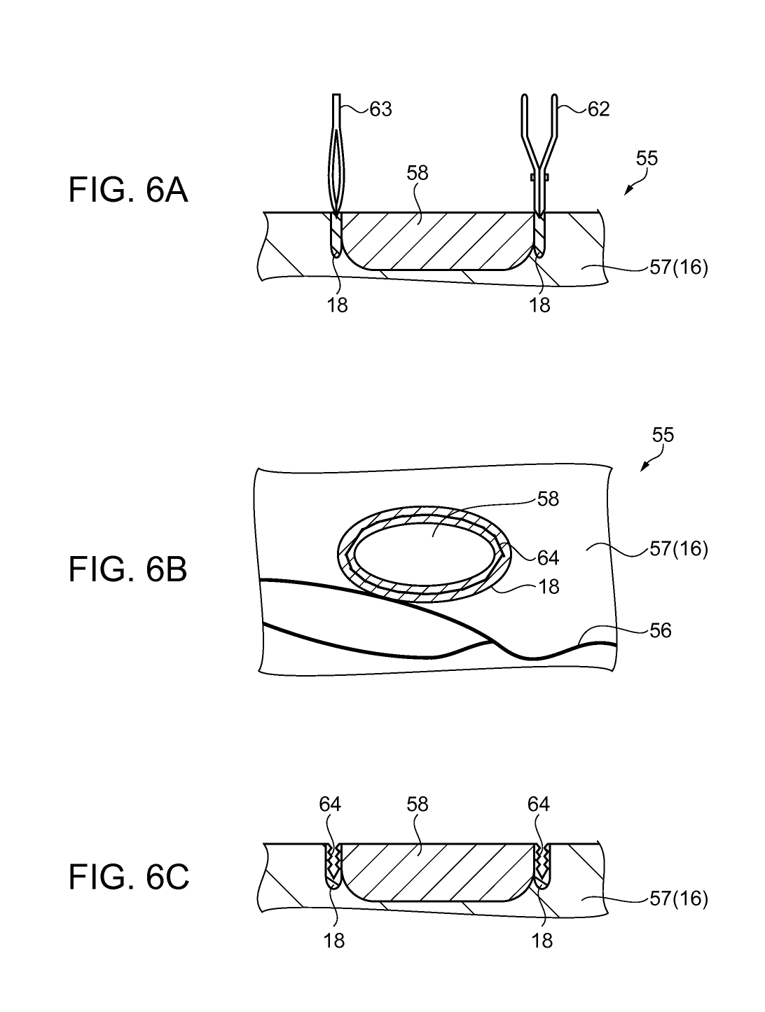

FIGS. 6A to 6C show the step of dissecting the soft tissue 18. As shown in FIG. 6A, the soft tissue 18 may be excised with scissors 62 as a dissection tool, or dissection may be carried out while expanding the excised site with tweezers 63 as a dissection tool. Consequently, a groove 64 is formed in the soft tissue 18, as shown in FIGS. 6B and 6C. The dissection tool may be an electric surgical apparatus such as an electric surgical knife that burns off a tissue with heat.

Moreover, in the liquid ejection device 1, the step of turning the cellular tissue 16 into the soft tissue 18 and the step of dissecting the soft tissue 18 are repeated, thus extirpating the tumor 58 from the normal tissues 57. Next, the pia mater 54, meninx 53 and the head soft tissue 51 are placed back in the original positions and stitched up. Then, the surgical operation ends.

As described above, the embodiment has the following advantageous effects.

(1) According to the embodiment, the scheduled dissection line 61 is set at the boundary between the normal tissues 57 and the tumor 58, and the liquid 12 is ejected at the site where the scheduled dissection line 61 is set. At the site where the liquid 12 is ejected, the liquid is poured inside while a part of the cellular tissue 16 is fractured. Thus, the distance between the cells expands and the tissue becomes the soft tissue 18. Then, the site where the liquid 12 is ejected is dissected with the dissection tool such as the scissors 62 and the tweezers 63. At the site where the distance between the cells is expanded and the tissue is softened, the tissue can be easily cut with the dissection tool. Therefore, the cellular tissue 16 can be dissected easily.

(2) According to the embodiment, the liquid 12 is ejected in the form of a pulse flow. Using the pulse flow enables increase in the pressure fluctuation applied to the cellular tissue 16. As the pressure fluctuation increases, fatigue fracture can occur more easily in the part that connects cells together. Therefore, the cellular tissue 16 can be softened easily.

(3) According to the embodiment, since the liquid 12 ejected from the nozzle 4 is ejected in a pulsed shape, the amount of water is smaller than in the case of continuously applying the liquid 12, and the liquid 12 can be poured more easily at the position where the tissues are to be split. Also, the liquid 12 is ejected with a strength that is not high enough to dissect the tissue with the liquid 12 alone. Therefore, a strong pulse flow is not applied for dissection, and unwanted excision or incision of tissues can be prevented. Moreover, in the soft tissue 18, since the spacing between the tissues is expanded, the dissection tool such as scissors or tweezers can enter into the dissection surface more easily. Also, since the tissues are softened at the position where the liquid 12 is poured to split the tissues, the dissection procedure can be carried out easily. Therefore, dissection can be achieved without damaging the blood vessels 56 and the surrounding normal tissues, as opposed to the case where the tumor 58 is to be dissected by force, posing a high risk of damaging the blood vessels 56 and the surrounding normal tissues.

### Second Embodiment

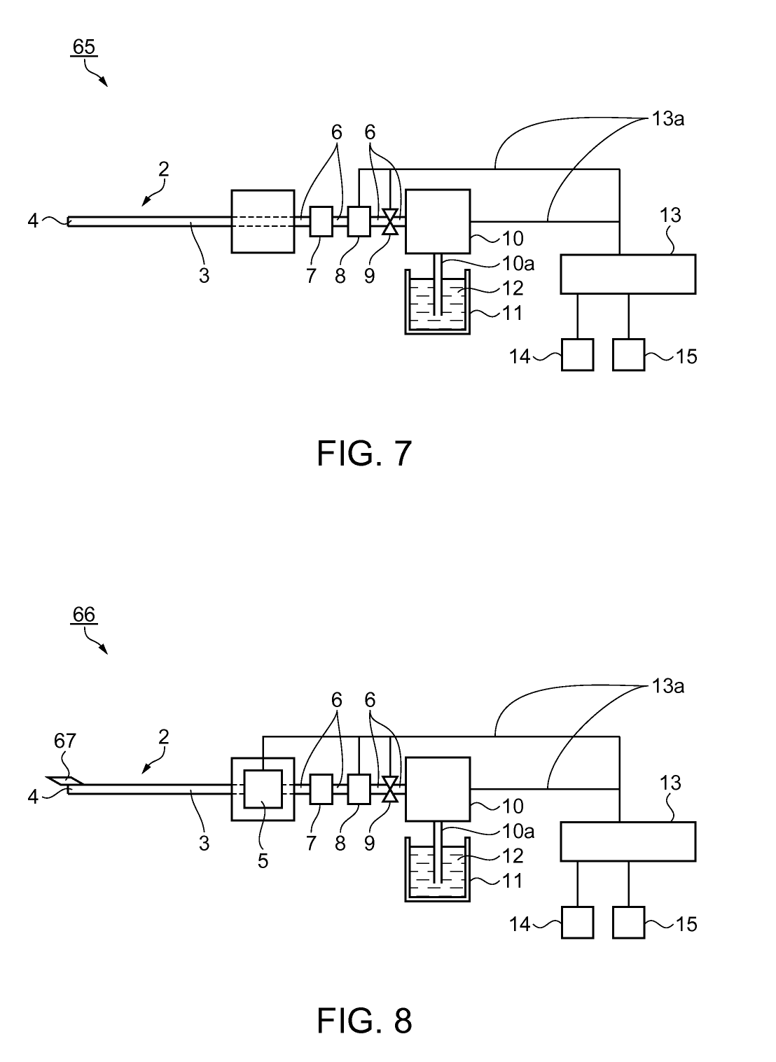

Next, an embodiment of a liquid ejection device will be described with reference to the block diagram of FIG. 7 showing the configuration of the liquid ejection device.

This embodiment is different from the first embodiment in that the pulsation applying part 5 of FIG. 1A is eliminated. The same features as in the first embodiment will not be described further.

That is, in this embodiment, in a liquid ejection device 65, the tube 6 is connected with the ejection tube 3, as shown in FIG. 7. As the surgical operator turns on the ejection switch 15, the liquid 12 is continuously ejected from the nozzle 4. Again, when the cellular tissue 16 can be turned into the soft tissue 18, a configuration without the pulsation applying part 5 may be employed. Since the pulsation applying part 5 and the pulsation control unit 50 can be omitted, the liquid ejection device 65 that can be easily manufactured is provided.

### Third Embodiment

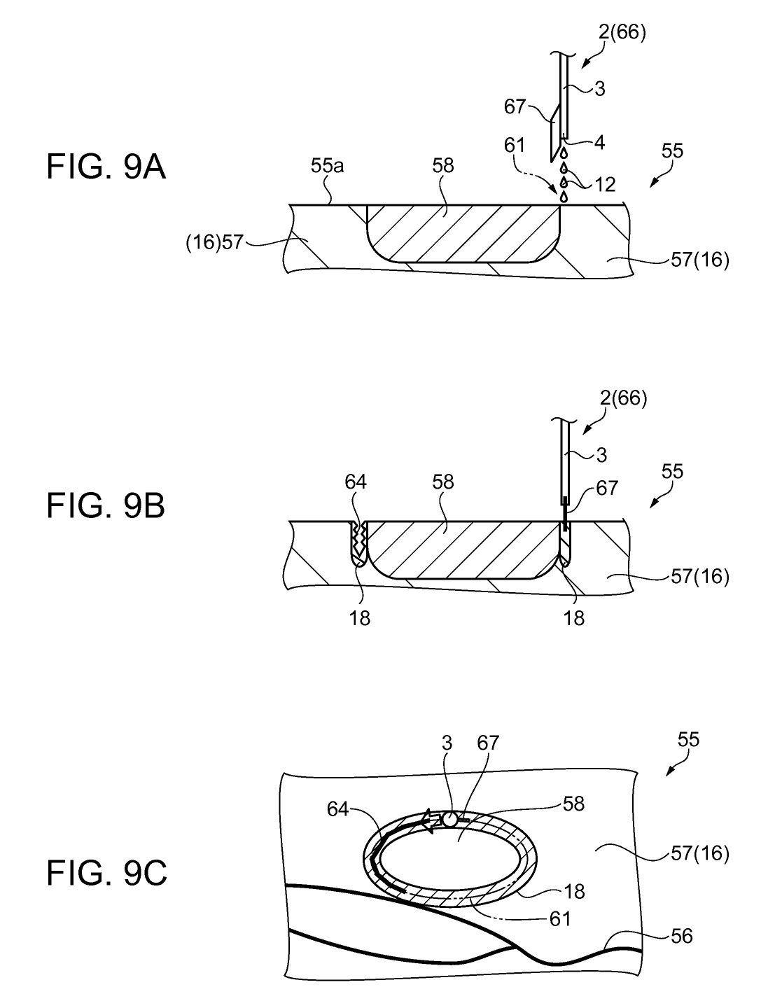

Next, an embodiment of a liquid ejection device will be described with reference to FIG. 8 and FIGS. 9A to 9C. FIG. 8 is a block diagram showing the configuration of the liquid ejection device. FIGS. 9A to 9C are schematic views for explaining a dissection method for dissecting a cellular tissue. This embodiment is different from the first embodiment in that an edge portion 67 as a dissection tool is installed near the nozzle 4. The same features as in the first embodiment will not be described further.

That is, in this embodiment, in a liquid ejection device 66, the edge portion 67 is installed on the ejection tube 3, as shown in FIG. 8. Thus, the surgical operator can eject the liquid 12 from the nozzle 4 toward the surface of the cellular tissue 16. Since the liquid ejection device 66 has the edge portion 67, the surgical operator can carry out both the formation of the soft tissue 18 and the dissection procedure of the soft tissue 18, using the liquid ejection device 66. Therefore, since the surgical operator need not hold a plurality of tools one after another, the cellular tissue 16 can be dissected with high operability.

The edge portion 67 is installed side by side with the nozzle 4. The end of the edge portion 67 is installed, protruding ahead of the end of the nozzle 4 in the direction in which the liquid 12 is ejected. Thus, the surgical operator can carry out the formation of the soft tissue 18 and the dissection procedure of the soft tissue 18 without changing the way of holding the liquid ejection device 66.

FIG. 9A shows the step of ejecting the liquid 12 to the cellular tissue 16 from the nozzle 4. As shown in FIG. 9A, the surgical operator ejects the liquid 12 toward the cellular tissue 16 from the nozzle 4. Then, the surgical operator operates the handpiece 2 to move the nozzle 4 along the scheduled dissection line 61. The edge portion 67 is installed at the end of the ejection tube 3. Since the length of the part of the edge portion 67 protruding ahead of the nozzle 4 is short, the liquid 12 can be ejected to the cellular tissue 16 without causing the edge portion 67 to contact the cellular tissue 16.

FIGS. 9B and 9C show the step of dissecting the soft tissue 18. As shown in FIGS. 9B and 9C, the soft tissue 18 is excised and dissected with the edge portion 67. The excision of the soft tissue 18 is carried out along the scheduled dissection line 61. Consequently, the groove 64 is formed in the soft tissue 18.

It should be noted that the embodiments are not limited to the above embodiments and that a person with ordinary skills in the art can make various changes and improvements without departing from the technical scope of the invention. Modifications will be described below.

### Modification 1

In the first embodiment, an example of dissecting the cellular tissue 16 of the cerebrum 55 is described. However, the application is not limited to the cerebrum 55 and a similar method to this embodiment may be used when dissecting the cellular tissue 16 at another site. The subject to be treated may be limited to animals excluding humans. Also, the cellular tissue 16 is not limited to a living body and may be a part of a corpse. In such cases, too, since the site of the soft tissue 18 can be easily cut with the dissection tool, the cellular tissue 16 can be easily dissected.

### Modification 2

In the third embodiment, the edge portion 67 is fixed to the ejection tube 3. A linear motion mechanism may be installed on the ejection tube 3 so that the linear motion mechanism can move the edge portion 67 in the longitudinal direction of the ejection tube 3. When the liquid 12 is ejected from the nozzle 4, the edge portion 67 may retreat into a place where the edge portion 67 does not protrude ahead of the nozzle 4. When the liquid 12 is ejected, the edge portion 67 can be prevented from contacting the cellular tissue 16 and therefore from making it difficult to operate the handpiece 2.

Moreover, when the dissection procedure of the soft tissues 18 is carried out, the linear motion mechanism causes the edge portion 67 to protrude ahead of the nozzle 4. Thus, since the edge portion 67 can be easily inserted in the soft tissue 18, the dissection of the soft tissue 18 can be made easier.

What is claimed is:

1. A cellular tissue dissection method comprising:
ejecting a liquid to a cellular tissue; and
dissecting, with a dissection tool, the cellular tissue at a site where the liquid is ejected.

2. The cellular tissue dissection method according to claim 1, wherein
the liquid is ejected in the form of a pulse flow.

3. The cellular tissue dissection method according to claim 1, wherein
the liquid is ejected from a nozzle of a liquid ejection device, and
the dissection tool is installed side by side with the nozzle, and the site where the liquid is ejected is dissected with the dissection tool.
